(19) United States
(12) Patent Application Publication
(10) Pub. No.: US 2011/0097264 A1
(43) Pub. Date: Apr. 28, 2011

(54) RADIOLABELING METHOD USING MULTIVALENT GLYCOLIGANDS AS HEPATIC RECEPTOR IMAGING AGENT

(75) Inventors: Mei-Hui Wang, Taoyuan County (TW); Wuu-Jyh Lin, Taoyuan County (TW); Chuan-Yi Chien, Taoyuan County (TW); Hung-Man Yu, Taoyuan County (TW); Reiko Takasaka Lee, Baltimore, MD (US); Yuan-Chuan Lee, Baltimore, MD (US)

(73) Assignee: Institute of Nuclear Energy Research Atomic Energy Council, Executive Yuan, Taoyuan County (TW)

(21) Appl. No.: 12/779,328

(22) Filed: May 13, 2010

Related U.S. Application Data
(60) Provisional application No. 61/264,898, filed on Nov. 30, 2009.

(30) Foreign Application Priority Data
Oct. 26, 2009 (TW) 098136143

(51) Int. Cl. A61K 51/08; C07K 5/083; C07F 5/00; A61K 51/04

(52) U.S. Cl. 424/1.69; 530/322; 534/10; 424/1.73

(57) ABSTRACT

A radiolabeling method using a multivalent glycoligand as hepatic receptor imaging agent is provided. The multivalent glycoligand-DTPA derivatives (In-111-DTPA-hexa lactoside and In-111-DTPA-tri-galactosamine glycoside) labeled with In-111 are used as hepatic receptor imaging agent. The effects of imaging of a hepatic receptor in different species are evaluated, the lowest specific radioactivity values of hepatic receptor imaging required in different species are discovered. Since the specificity of the human ASGPR closely resembles that of the mouse. This kind of radiolabelling method, agent and related study about specific radioactivity could be used in clinical trial in the future.

### BACKGROUND OF THE INVENTION

1. Field of the Invention

The present invention relates to a radiolabeling method using multivalent glycoligands as hepatic receptor imaging agent, which is used to evaluate the effect of imaging of a hepatic receptor in different species, and the lowest specific radioactivity values of hepatic receptor imaging required by different species.

2. Related Art

Asialoglycoprotein receptor (ASGPR) in the liver is known to specifically bind to saccharide chains having Gal or GalNAc on an end, thus it is desirable to develop saccharide chains having a Gal or GalNAc end to serve as hepatic receptor imaging agent. The hepatic receptor imaging agent has the following utilities in the industry.
1. As liver transplantation often fails due to too severe liver damage caused by transient hypoxia-reperfusion, whether the liver transplantation is successful or not can be immediately known through hepatic receptor imaging after the transplantation.
2. Hepatic receptor imaging is indicative of actual liver function. After binding with ASGPR, glycopeptides or glycoproteins with Gal and GalNAc end enters hepatocytes through receptor-mediated endocytosis. When liver lesion occurs, the hepatic receptor is reduced, and the imaging level will be reduced. Thus, the actual liver function can be evaluated by the imaging level theoretically.
3. Hepatic receptor imaging can be used to evaluate the anti-hepatitis and anti-fibrotic effects of Chinese herbal medicines.
4. The hepatic receptor imaging agent has highly specific liver targeting property, and can effectively carry medicines to be accumulated into liver at a concentrated dosage, so that not only the used dosage and treatment cost can be significantly reduced, but also the generation of side effects can be effectively reduced.
5. The hepatic receptor imaging agent is highly safe, and can be used as gene delivery vector for liver without any unnecessary allergic immune response.
6. The hepatic receptor imaging agent is a good tool to observe the ASGPR specificity between mammals, which will benefit to study if the ASGPR is universal or not.

Presently, the peptides or proteins to be polymerized with saccharide groups that have been disclosed in documents and patents include albumin, tyrosine-glutamyl-glutamic acid (YEE), tyrosine-aspartyl-aspartic acid (YDD), and tyrosine-glutamyl-glutamyl-glutamic acid (YEEE).

Tc-99m-Galactosyl-Serum-Albumin (Tc-99m GSA) is known as a hepatic receptor imaging agent. YEE and YDD are firstly set forth by Lee et al (JBC 258:199-202, 1983), and YEEE is an improved invention by Chen et al (Taiwan Patent No. TW1240002, 2000). In 1983, Lee et al set forth that the binding force between galactosamine peptide with two chains in series and hepatocyte is 1000 times of that of galactosamine peptide with a single chain, and the binding force between galactosamine peptide with three chains in series and hepatocyte is 10⁶ times of that of galactosamine peptide with a single chain. (JBC 258:199-202, 1983). It is necessary to find a scaffold having at least three functional groups to polymerize three galactosamine chains together, for which a polymerized amino acid, i.e., peptide, is useful, for example, glutamyl-glutamic acid (EE, in which glutamic acid is represented as E), aspartyl-aspartic acid (DD, in which aspartic acid is represented as D), and lysine-lysine (KK, in which lysine is represented as K). Both EE and DD have three COOH functional groups being exposed and can thus be jointed with three galactosamine peptides having a certain length. As for KK, it has three amino groups and one COOH functional group, with the three amino groups being not easily linked to the saccharide chains, so it has not been used to develop hepatic receptor imaging agent till now.

In order to facilitate iodine isotope labeling, EE and DD are connected with Y (tyrosine), allowing in vivo imaging or cell receptor binding test. However, for the iodine labeling of YEE or YDD, it is necessary to add an oxidant, such as chloramine T, Iodobead, or Iodogen. Further, in case of in vivo imaging, it is necessary to remove the oxidant by purification at the end of the reaction, because the oxidants are toxic to human body.

The ideal radiolabeling of hepatic receptor imaging agent is one mole multivalent glycoligand being jointed with one mole radioisotope, which is difficult in practice. Even for the saccharide chains having a similar structure, the radiochemical properties may not be completely identical. Besides adjusting the ligand/In-111 molar ratio, buffer selection, and reaction temperature, the specific radioactivity required for radiolabeling needs to be determined through animal experiments. According to the study on ASGPR specificity in different species by Park et al in 2004 (JBC 279:40954-40959, 2004.), the ASGPR specificity in human is close to that in mice. Therefore, a study on the minimal specific radioactivity of hepatic receptor imaging required for mice is helpful to assess the specific radioactivity to be required in human body test.

### SUMMARY OF THE INVENTION

Accordingly, in order to solve the problems, the present invention provides hepatic receptor imaging agents, DCM-Lys(GahGalNAc)₃ and AHA-Asp[DCM-Lys(ahLac)₃]₂, and a molecular imaging technique to discuss the specific radioactivity minimally required by different species. In the design according to the present invention, lysine is further modified, that is, the α-amino group of lysine and glycolic acid are subjected to reductive alkylation, so that N carries two CH₂COOH, together with one COOH and one NH₂ of lysine itself, three saccharide chains can be polymerized. Furthermore, the free amino group can be further bridged with DOTA and DTPA to form a precursor of the hepatic receptor imaging agent suitable for In-111, Tc-99m, Ga-68, and Gd labeling. Compared with iodine labeling, the In-111, Ga-68, and Tc-99m labeling have the advantage of being free of oxidant such as chloramine T, Iodobead, and Iodogen, thus have low toxicity. Therefore, a new liver targeting drug can be provided that is different from YEE and YDD but is suitable for In-111 or Tc-99m labeling. Additionally, through discussing the minimal specific radioactivity of hepatic receptor imaging required by different species, the specific radioactivity to be required in human body test in the future can be assessed.

The present invention provides a method of radiolabeling a novel hepatic receptor imaging agent of six lactose glycoside chains with In-111. A trivalent radioisotope In-111 is added into DTPA-hexa lacto side-DCM-lysine (DTPA-hexa-lactoside-dicarboxymethyl-L-lysine), and reacted under shaking at room temperature for 15 min. The optimal specific activity of the hepatic receptor imaging agent is 2.5×10¹⁰ Bq/mg, and the radiochemical purity of the labeled agent is up to above 99%. When imaging is performed at this specific radioactivity, the required dose is merely 200 nCi/g, i.e. the required dose is 4 uCi for a 20 g mice. Since the liver of an adult weighs 1000 times of that of a 20 g mice, we figure out the imaged radioactivity for an adult is about 4 mCi. In the future, DTPA-Lactoside-DCM-lysine can be made in lyophilized dosage form, which is beneficial for sale abroad. Because the trivalent radioisotope In-111 is added into DTPA-hexa-lactoside-DCM-lysine directly, the process is simple without purification, has very low toxicity and very high safety.

In another aspect, the present invention provides a method of radiolabeling a novel hepatic receptor imaging agent of three galactosamine glycoside chains with In-111. A trivalent radioisotope In-111 is added into DTPA-trivalent GalNAc glycoside-DCM-lysine (i.e., DTPA-tri-GalNAc glycoside-dicarboxymethyl-L-lysine) and reacted under shaking at a required temperature of 90° C. or 100° C. for 30 min, to give a product with a specific activity of 3.4×10⁸ Bq/mg. However, if the specific activity is lower than 1.7×10⁸ Bq/mg, the hepatic receptor imaging agent can merely used in rat imaging, but not in mice imaging.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The present invention will become more fully understood from the detailed description given herein below for illustration only, and thus are not limitative of the present invention, and wherein:

FIG. 1 is a structural representation of a liver targeting drug;

FIG. 2 is an instant thin-layer chromatography (ITLC) spectrum of In-111-DTPA-lactoside, in which the radiochemical purity is up to 99%, and the specific radioactivity is 2.5×10¹⁰ Bq/mg;

FIG. 3 is a distribution data graph of In-111 hexa-lactoside in an organism (mouse);

FIG. 4 is a whole-body autoradiography image of an organism (mouse);

FIG. 5 is a SPECT/CT image and tomography by a hepatic receptor imaging agent;

FIG. 6 is a relationship diagram of hexa lactoside/In-111 molar ratio and radiochemical yield;

FIG. 7 shows the absorption of In-111 hexa lactoside by murine hepatocytes of various species;

FIG. 8 is liver absorption curves of rat and mice on sequence In-111-hexa lactoside;

FIG. 9A is a SPECT/CT image of In-111 DTPA-tri-GalNAc glycoside of different specific radioactivity in molecular imaging in mouse, in which the specific radioactivity is 1.1×10⁹ Bq/mg;

FIG. 9B is a SPECT/CT image of In-111 DTPA-tri-GalNAc glycoside of different specific radioactivity in molecular imaging in mouse, in which the specific radioactivity is 3.4×10⁸ Bq/mg;

FIG. 9C is a SPECT/CT image of In-111 DTPA-tri-GalNAc glycoside of different specific radioactivity in molecular imaging in mouse, in which the specific radioactivity is 1.7×10⁸ Bq/mg;

FIG. 10A is a SPECT/CT image of In-111 DTPA-tri-GalNAc glycoside of different specific radioactivity in molecular imaging in rat, in which the specific radioactivity is 1.7×10⁸ Bq/mg; and

FIG. 10B is a SPECT/CT image of In-111 DTPA-tri-GalNAc glycoside of different specific radioactivity in molecular imaging in rat, in which the specific radioactivity is 3.7×10⁷ Bq/mg.

### DETAILED DESCRIPTION OF THE INVENTION

The features and implementation of the present invention are described in detail with preferred embodiments below.

I Design of Novel Liver Targeting Drug

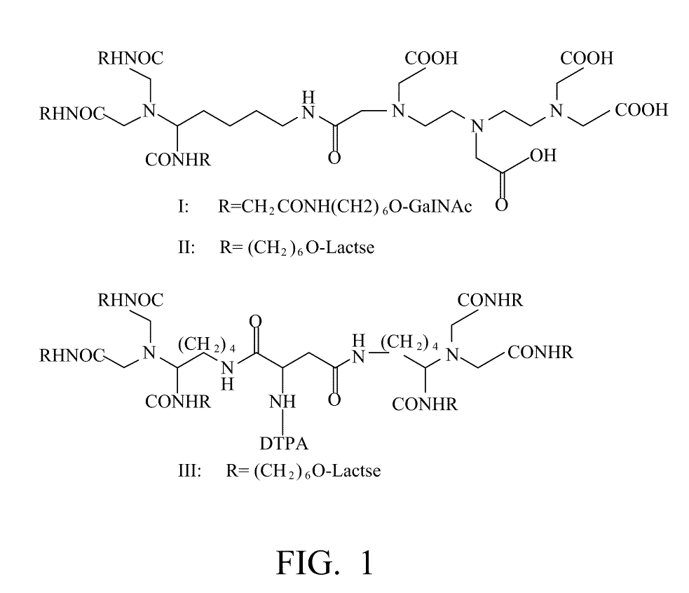

According to the present invention, ε-benzyloxycarbonyl-α-dicarboxylmethyl-L-lysine (Z-DCM-Lys) is used as a new basic structure to be connected to aminohexyl β-GalNAc (ah-GalNAc), glycyl-aminohexyl β-GalNAc (Gah-GalNAc), or aminohexyl Lac (ah-Lac), so as to form a three-chain glycopeptide. As the binding strength of the lactose glycoside and the ASGPR is not as strong as that of the galactosamine glycoside, when the lactose glycoside is connected in series, two molecules of three-chain lactose glycoside will be further connected in series through aspartic acid or glutamic acid. For example, two molecules of ε-Z-α-DCM-Lys(ah-Lac)₃ is further connected together through aminohexanoyl aspartic acid (AHA-Asp) to form AHA-Asp[DCM-Lys(ah-Lac)₃]₂ (hereafter simply referred to as hexa-Lactoside). The free amino end of the hexa-Lactoside can react with DTPA anhydride in sodium carbonate solution to form a DTPA derivative of AHA-Asp[DCM-Lys(ah-Lac)₃]₂, as shown in FIG. 1.

II Analysis of Binding Strength of Saccharide Chain Peptide and Murine Hepatocyte

With Eu-asialo-orosomucoid (Eu-ASOR) as reference material, the binding strength of saccharide chain peptide and murine hepatocyte is determined by comparing whether the binding degree of saccharide chain peptides, such as DCM-Lys(ah-GalNAc)₃, DCM-Lys(Gah-GalNAc)₃, DCM-Lys(ah-Lac)₃, AHA-Asp[DCM-Lys(ah-Lac)₃]₂ with murine hepatocyte is higher than that of Eu-ASOR or not, in which the binding degree is expressed by IC₅₀ (concentration of 50% inhibition), and the lower the IC₅₀ is, the higher the binding degree is. The murine hepatocyte (from Lonza Biotechnology Company, Maryland) is plated in a 24-well plate in advance, and the reaction occurs in each well, into which (i) Eu-ASOR 10 nM (ii) hepatocyte basic medium with 5 mM calcium chloride, and (iii) five different concentrations of saccharide chain peptide of 1 uM-0.8 nM are added. After culturing under shaking for 1 hr, the substance that has not been bound to hepatocyte is removed by washing with the hepatocyte basic medium containing calcium chloride. Time-resolved fluorescence spectroscopy is preformed, that is, an enhancement solution (15

uM β-naphthoyl trifluoroacetone, 50 uM tri-n-octyl-phosphine oxide, 0.1 M potassium hydrogen phthalate, 0.1% Triton X-100 in 0.1 M acetic acid, pH 3.2) is added. The enhancement solution reacts with Eu³⁺ to form a Eu chelate, which can emit a light of 615 nm when being excited at 340 nm. With the logarithm of the concentration of saccharide chain peptide as X axis, the emitted fluorescence value as Y axis, the fluorescence value without adding glycopeptide being set as 100%, the IC₅₀ of each saccharide chain peptide can be calculated accordingly. As shown in Table 1, it can be known from the data that, the binding of AHA-Asp[DCM-Lys(ah-Lac)₃]₂ and ASGPR can reach the same binding strength as that of YEE or YDD, but the binding of DCM-Lys(Gah-GalNAc)₃ and ASGPR is 10 times of that of YEE or YDD.

TABLE 1
Comparison of binding strength of various saccharide chains and
murine hepatocyte

| Compounds | IC50 (nM) |
|---|---|
| YEE(ahGalNAc)₃ | 10 nM |
| YDD(GahGalNAc)₃ | 10 nM |
| DCM-Lys(ahGalNAc)₃ | 10 nM |
| DCM-Lys(GahGalNAc)₃ | 1 nM |
| AHA-Asp[DCM-Lys(ahLac)₃]₂ | 10 nM |

III Method of Radiolabeling Hepatic Receptor Imaging Agent

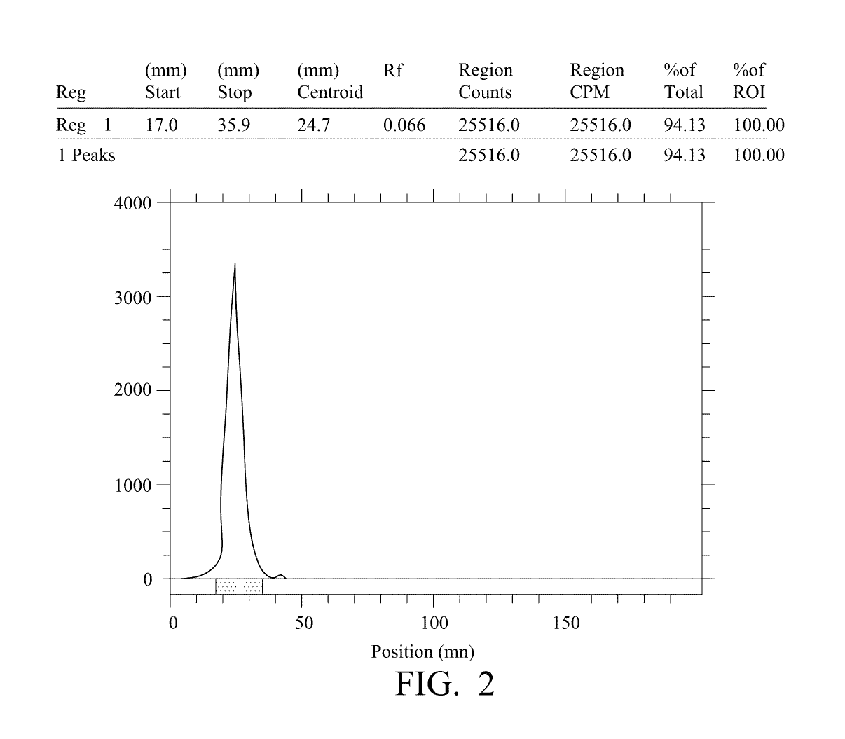

30 μCi In-111(6×10⁻¹³ moles) is reacted with 43.8 ng DTPA-hexa-lactoside (1.2×10⁻¹¹ moles) for 15 min in 0.1 M citric acid (pH 2.1), and the radiochemical purity of In-111-DTPA-lactoside is determined by radio-ITLC (instant thin-layer chromatography). Briefly, a sample of the reaction product above is spotted on an ITLC-SG strip, and is placed in a developing chamber with 10 mM citrate buffer (pH 4) for development. When the liquid level reaches the development end point, the strip is taken out, and placed in a fume chamber for drying, and then scanned with a radio-TLC analyzer, to analyze Rf value (retention factor, which is distance traveled by the analyte divided by distance traveled by the mobile phase). In-111-hexa-lactoside will stay around its origin, and free In-111 and In-111 DTPA will stay at the front of the developing phase. Individual spectrum is plotted and integrated, as shown in FIG. 2.

IV Bio-Distribution

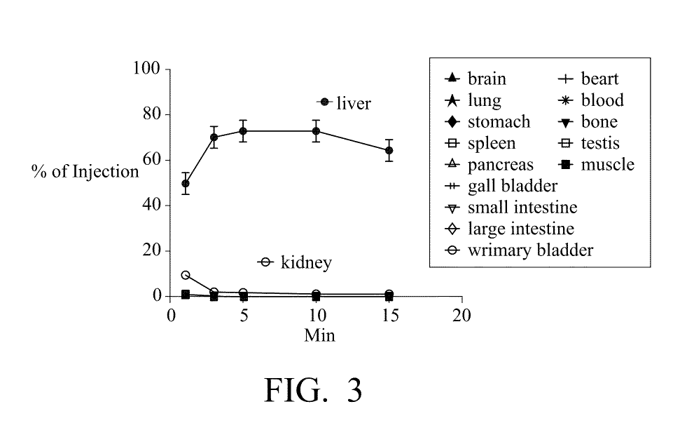

In-111 hexa-lacto side (200 nCi/g) is injected via tail vein into mice, and the mice are sacrificed at 1 min, 3 min, 5 min, 10 min, 15 min, 1 hr, 24 hr by cervical dislocation, and organs in body are taken out to collect biological samples of mice, including whole blood, brain, muscle (thigh), bone, stomach, spleen, pancreas, small intestine, large intestine, lung, heart, kidney, gallbladder, liver, bladder, urine, etc. The samples are weighed and then placed in a measuring tube. The organs and the standards are placed in a Gamma counter (Cobra II Auto-Gamma Counter, PACKARD, U.S.A) for measurement, to calculate the percentage of injected dose per organ (% ID). The experimental data is presented as mean±standard error of mean (mean±SEM), and time-activity curve is plotted, thereby the actual radiation dose distribution in the body is calculated, as shown in FIG. 3. It can be seen from the bio-distribution data graph that, nearly 80% of the activity is accumulated in liver, and no radioactivity is accumulated in other organs except urine. As 75% of the blood flow of mice is concentrated in the kidney, part of the radioactivity is inevitably distributed in the urine. If the distribution in urine is ignored, the distribution in liver should be nearly 100%, which is sufficient to prove the liver targeting characteristics.

V Whole-Body Autoradiography

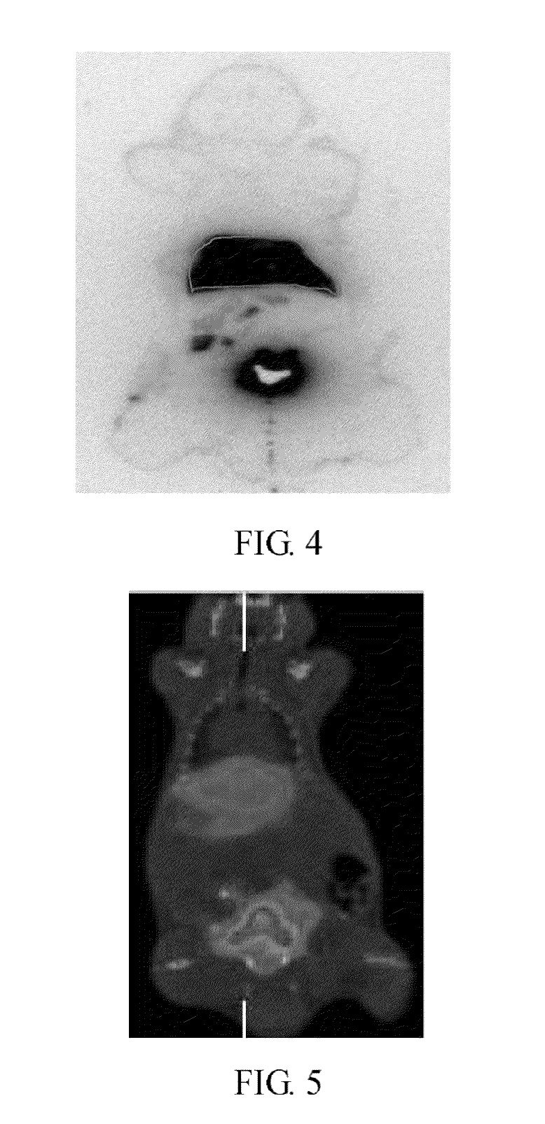

In-111 hexa-lactoside (200 nCi/g) is injected via tail vein into mice, and after 15 min of distribution, whole-body freezing microtomy is performed (CM 3600, Leica Instrument, Germany) to obtain sections of 20-30 μm in thickness. The radioactivity is exposed onto X-ray films. A selected section is placed on an IP plate and then placed into a cassette, and exposed with X-ray films at −20° C., thus the radioactivity on the organ will be imaged on the corresponding position on the X-ray film, and the image strength is in proportion to the radioactivity strength on the organ (autoradiography). The image is analyzed with BAS-1000, Fuji Film Image reader, and Image Gauge, to get whole-body autoradiography image, as shown in FIG. 4. The autoradiography image is consistent with the bio-distribution data, that is, radioactivity is merely present in liver and urine.

VI SPECT/CT Image and Tomography by Hepatic Receptor Imaging Agent

In-111 hexa-lactoside (200 nCi/g) is injected via tail vein into mice, SPECT/CT (Gamma Medica Idea (GMI) X-SPECT) is performed immediately after injection, and the imaging lasts for 15 min with a medium energy parallel-hole collimator. In imaging, the animals for experiment are anaesthetized by isoflurane, and after imaging, the SPECT/CT image fusion is preformed, as shown in FIG. 5. The SPECT/CT image is consistent with the biodistribution and autoradiography image data, that is, radioactivity is merely present in liver and urine. Therefore, the position of liver is selected to quantify the image strength in the liver.

VII Study on the Effect of Hexa Lactoside/In-111 Molar Ratio on Radiochemical Yield

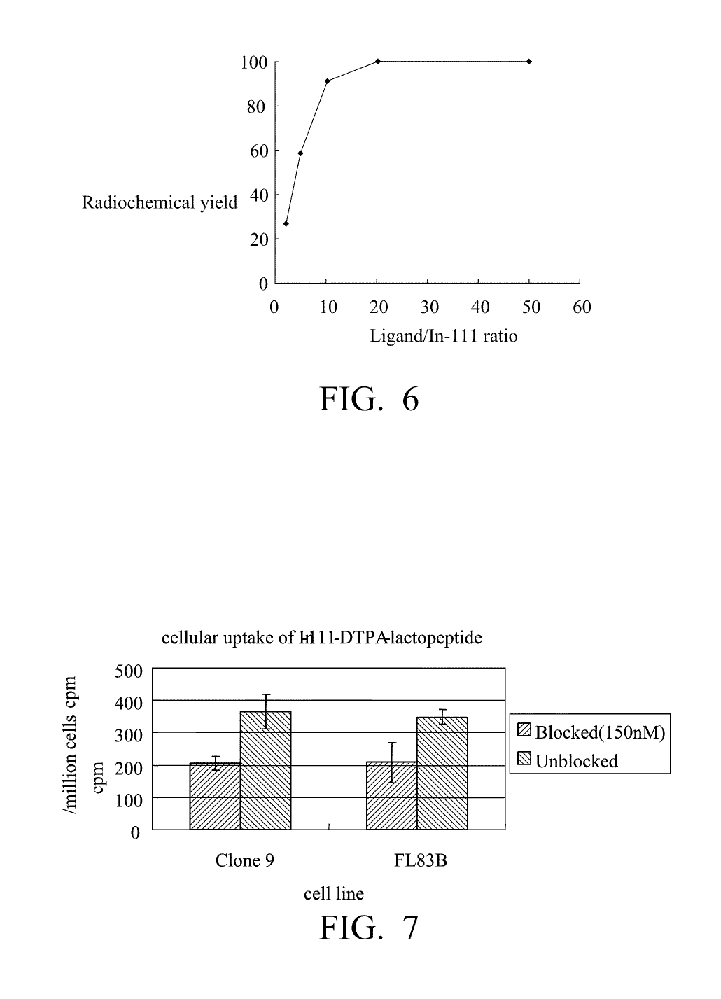

DTPA-hexa-lactoside of different concentrations are placed in microcentrifuge tubes, 0.1 M citric acid (pH 2.1) and In-111-InCl₃ solution are added, the radioactivity is about 30 μCi, and the microcentrifuge tubes are gently shaken to make the contents mixed completely. The labeling reaction is performed at room temperature for 15 min and then sampled to analyze the radiochemical purity of In-111-DTPA-hexa-lactoside with radio-ITLC. The relationship diagram of hexa lactoside/In-111 molar ratio and the radiochemical yield is as shown in FIG. 6, and the data indicates that when the hexa lactoside/In-111 molar ratio is higher than 20, a radiochemical yield of up to higher than 99% can be obtained, and at this time, the specific radioactivity is 2.5×10¹⁰ Bq/mg.

VIII Absorption of In-111 Hexa-Lacoside by Hepatocyte

Clone 9 is rat hepatocyte, FL83B is mouse hepatocyte. 1×10⁶ cells/cc Clone 9, and FL83B cells are plated in a 6-well culture plate, 1 μCi In-111 hexa-lactoside is added to react at 37° C. for 1 hr, and after removal of the supernatant, washed 2× with phosphate buffer solution. The cells are removed by adding trypsin, and also washed 2× with phosphate buffer solution. The radioactivities absorbed by the cells is measured with a Gamma counter (Cobra II Auto-Gamma Counter, PACKARD, U.S.A). The above steps are repeated, that is, 1×10⁶ cells/cc Clone 9 and FL83B cells are plated in a 6-well culture plate, 150 nM hexa-lactoside is firstly added to react for 1 hr, and then 1 μCi In-111 hexa-lactoside is added to react for 1 hr at 37° C., and after removal of the supernatant, washed 2× with phosphate buffer solution. The cells are removed by adding trypsin, and also washed 2× with phosphate buffer solution. The radioactivity absorbed by the cells is measured with a Gamma counter (Cobra II Auto-Gamma Counter, PACKARD, U.S.A). The results are shown in FIG. 7. The same number of mouse and rat hepatocytes has the same absorption on In-111 hexa-lactoside. If the hepatocytes are occupied by a high concentration (150 nM) of hexa-lactoside firstly, almost all the absorption of In-111 hexa-lactoside by the hepatocytes of various species is background value.

IX Establishment of Liver Absorption Curve of Sequence In-111-Hexa-Lactoside Glycopeptide

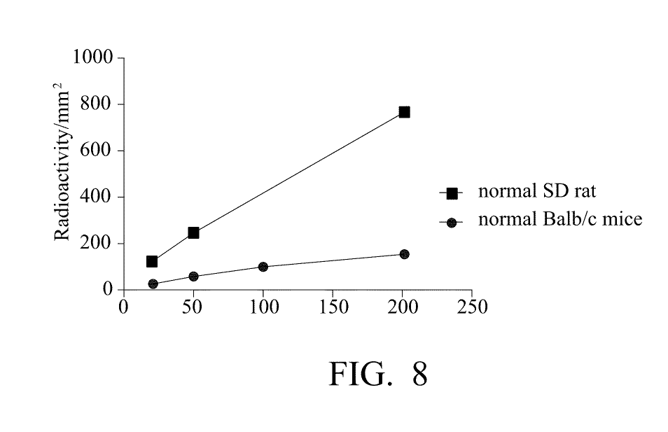

In-111-hexa-Lactoside is injected via tail vein into rats and mice at dosages of 20 nCi/g, 50 nCi/g, 100 nCi/g, and 200 nCi/g, SPECT/CT imaging is performed for 15 min, and quantitative analysis and tomography experiments are performed as well. A liver scope is selected to quantify the image strength, and the curves of activity dose of the sequence and liver absorption radiation dose are plotted. The liver absorption curves of rats and mice on sequence In-111-hexa lactoside are as shown in FIG. 8. It can be seen from the results that, the absorption per unit liver area of rats is higher than that of mice. Since the absorption of In-111 hexa lactoside by the same number of hepatocytes of rats and mice is the same. We inferred ASGPR per unit area of rats and mice are not the same was due to the density of ASGPR in rats is higher than that in mice. This is good example that our labeling method and related agent could be used to observe the ASGPR specificity between mammals, which may be useful to study if the ASGPR is universal or not.

X Study on the Effect of Tri-Galactosamine Glycoside and In-111 Molar Ratio on Radiochemical Yield at Different Temperatures

DTPA-tri-GalNAc glycoside (molecular weight 1474 Da) of different concentrations are placed in microcentrifuge tubes, 0.1 M citric acid (pH 2.1) and In-111-InCl₃ solution are added, the radioactivity is about 30 μCi (i.e. 1.1×10⁶ Bq; 6.4×10⁻¹³ mole), and the microcentrifuge tubes are gently shaken to make the contents mixed completely. The labeling reaction is performed at room temperature, 90° C., or 100° C. for 30 min and then sampled to analyze the radiochemical purity of In-111-DTPA-tri-GalNAc glycoside with radio-ITLC, and the results are as shown in Table 2.

TABLE 2
Relationship of tri-galactosamine glycoside/In-111 molar ratio and radiochemical
yield at different temperatures

| Tripolygalactosamine | | | | Specific |
|---|---|---|---|---|
| chain and In-111 | Radiochemical yield (%) | | | radioactivity |
| molar ratio | 30 min @ RT | 30 min @ 90° C. | 30 min @ 100° C. | Bq/mg |
| 10593 | 43 | — | — | 4.6 × 10 [symbol] |
| 10593 | 44 | 83 | — | 9.3 × 10 [symbol] |
| 5000 | 54 | 87 | — | 2.0 × 10 [symbol] |
| 3300 | — | — | 90 | 3.4 × 10 [symbol] |
| 100 | 64 | 82 | — | 9.8 × 10 [symbol] |
| 50 | 66 | 83 | — | 2 × 10 [symbol] |
| 20 | 76 | 80 | — | 4.8 × 10 [symbol] |
| 10 | 74 | 78 | — | 9.4 × 10 [symbol] |

[symbol]

indicates data missing or illegible when filed

XI Study on Molecular Imaging in Mouse with In-111 DTPA-Tri-GalNAc Glycoside of Different Specific Radioactivities

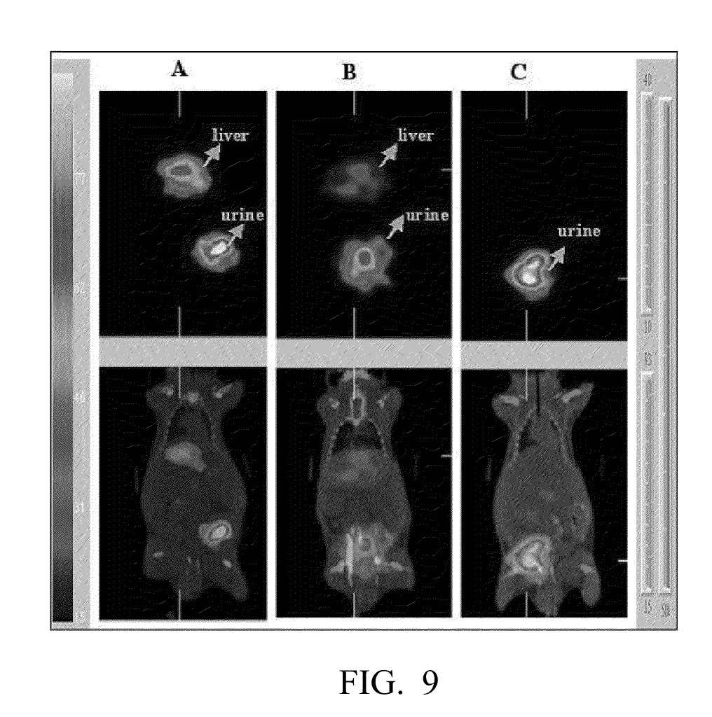

In-111 DTPA-tri-GalNAc glycoside of different specific radioactivities are injected via tail vein into mice, SPECT/CT(Gamma Medica Idea (GMI) X-SPECT) is performed immediately after the injection, and the imaging lasts for 15 min with a medium energy parallel-hole collimator. In imaging, the animals for experiment are anaesthetized by isoflurane, and after imaging, the SPECT/CT image fusion is preformed, as shown in FIGS. 9A, 9B, and 9C. The specific radioactivity from an image in FIG. 9A is 1.1×10⁹ Bq/mg, the specific radioactivity from an image in FIG. 9B is 3.4×10⁸ Bq/mg, and the specific radioactivity from an image in FIG. 9C is 1.7×10⁸ Bq/mg. The result indicates that for SPECT/CT imaging of mice with In-111 DTPA-tri-GalNAc glycoside, the specific radioactivity must be higher than 3.4×10⁸ Bq/mg.

XII Study on Molecular Imaging in Rat with In-111 DTPA-Tri-GalNAc Glycoside of Different Specific Radioactivities

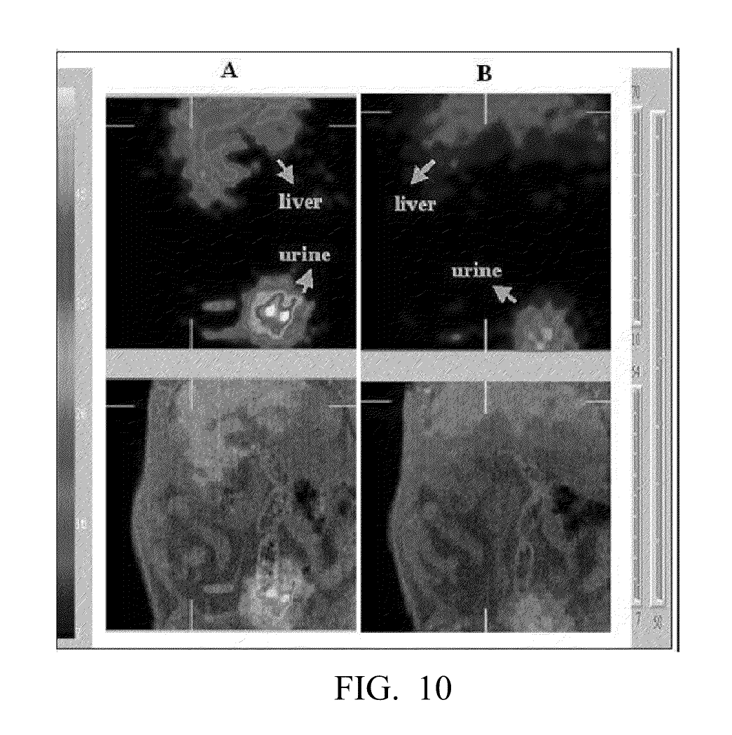

In-111 DTPA-tri-GalNAc glycoside of different specific radioactivities are injected via tail vein into rats, SPECT/CT(Gamma Medica Idea (GMI) X-SPECT) is performed immediately after the injection, and the imaging lasts for 15 min with a medium energy parallel-hole collimator. In imaging, the animals for experiment are anaesthetized by isoflurane, and after imaging, the SPECT/CT image fusion is preformed, as shown in FIGS. 10A and 10B. The specific radioactivity from an image in FIG. 10A is 1.7×10⁸ Bq/mg, and the specific radioactivity from an image in FIG. 10B is 3.7×10⁷ Bq/mg. The result indicates that even when the specific radioactivity of In-111 DTPA-tri-GalNAc glycoside is lower than 3.7×10⁷ Bq/mg in SPECT/CT imaging in rat, a clear image can be obtained.

Although the specific embodiments have been illustrated and described above, it will be apparent to those skilled in the art that various modifications and variations can be made in the present invention without departing from the scope or spirit of the invention. Furthermore, the present invention is not limited to the particular forms, and covers all modifications and variations of this invention provided they fall within the scope of the following claims and their equivalents.

In view of the above, in terms of its general combination and features, the present invention has not been found in similar products, and has not been disclosed before its filing date. It indeed meets the requirements of a patent and we thus propose this application according to the provisions of the patent law.

What is claimed is:

1. A radiolabeling method using a multivalent glycoligand as hepatic receptor imaging agent, formed by reacting a multivalent glycoligand-DTPA derivative with In-111 at room temperature or under heating for 30 min.

2. The multivalent glycoligand according to claim 1, wherein serial concentrations of the multivalent glycoligands react with a fixed amount of In-111 to get specific radioactivity of sequence In-111-DTPA-multivalent glycoligands.

3. The multivalent glycoligand according to claim 1, wherein the multivalent glycoligand is In-111-DTPA-hexa lactoside (In-111-DTPA-AHA-Asp[DCM-Lys(ah-Lac)₃]₂).

4. The multivalent glycoligand according to claim 1, wherein the multivalent glycoligand is In-111-DTPA-tri-galactosamine glycoside (In-111-DTPA-DCM-Lys(Gah-GalNAc)₃).

5. The multivalent glycoligand according to claim 3, wherein the In-111-DTPA-hexa lactoside is reacted at room temperature, the most preferred molar ratio of hexa lactoside and In-111 is 20, the labeling yield is above 99%, no addition of oxidant and purification are required in the labeling process, and the specific radioactivity is 2.5×10¹⁰ Bq/mg.

6. The multivalent glycoligand according to claim 3, wherein the In-111-DTPA-hexa lactoside is absorbed by hepatocytes, and in presence of the same number of hepatocytes, the hepatocyte absorption in rat and mice is the same.

7. The multivalent glycoligand according to claim 3, wherein the In-111-DTPA-hexa lactoside is used to perform whole-body SPECT/CT imaging in rat and mice, in order to quantify the absorption of the In-111-DTPA-hexa lactoside per unit area, the ASGPR per unit area in rat and mice is not the same, and the ASGPR density of rat is higher than that of mice.

8. The multivalent glycoligand according to claim 4, wherein the In-111-DTPA-tri-galactosamine glycoside is reacted at a temperature higher than 90° C., and the labeling yield is up to above 80%.

9. The multivalent glycoligand according to claim 4, wherein the specific radioactivity of the In-111-DTPA-tri-galactosamine glycoside is required to be higher than 3.4×10⁸ Bq/mg when being used for imaging in mice, and when being used for imaging in rat, even the specific radioactivity is lower than 3.7×10⁷ Bq/mg, a clear image is obtained.
